(19) United States
(12) Patent Application Publication
(10) Pub. No.: US 2010/0210668 A1
(43) Pub. Date: Aug. 19, 2010

(54) COMPOSITION AND METHOD FOR TREATMENT OR PREVENTION OF BENIGN PROSTATIC HYPERPLASIA AND LOWER URINARY TRACT SYMPTOMS

(75) Inventors: Seul-Min Choi, Gyeonggi-do (KR); Ju-Mi Kim, Gyeonggi-do (KR); Kyung-Koo Kang, Gyeonggi-do (KR); Byoung-Ok Ahn, Gyeonggi-do (KR); Moo-Hi Yoo, Seoul (KR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: DONG-A PHARM. CO., LTD., Seoul (KR)

(21) Appl. No.: 12/680,777

(22) PCT Filed: Sep. 24, 2008
(86) PCT No.: PCT/KR08/05669

§ 371 Date: Mar. 30, 2010

(30) Foreign Application Priority Data
Oct. 2, 2007 (KR) 10-2007-0099016
Feb. 27, 2008 (KR) 10-2008-0017768

(51) Int. Cl. A61K 31/519; A61P 13/02

(52) U.S. Cl. 514/262.1

(57) ABSTRACT

The present invention relates to a composition and a method for treating or preventing benign prostatic hyperplasia (BPH) and lower urinary tract symptoms (LUTS) without showing the side effect, by dramatically relaxing the smooth muscle in prostate and bladder.

### TECHNICAL FIELD

The present invention relates to a combination therapy for benign prostatic hyperplasia (BPH) and lower urinary tract symptoms (LUTS). More specifically, the present invention relates to a composition and a method for treating or preventing benign prostatic hyperplasia and lower urinary tract symptoms without showing the side effect, by relaxation of smooth muscle in prostate and bladder.

### BACKGROUND

Benign prostate hyperplasia (BPH) is a disease with a symptom of prostate enlargement caused by abnormal growth of prostatic cell. BPH occurs frequently in male in fifties or older. The prevalence rate of BPH increases sharply as the age increases, thereby affecting highly quality of life.

In general, BPH is accompanied by lower urinary tract symptoms of which the cause are anatomical obstruction by the prostate and functional obstruction by α1-receptor to contract the smooth muscle in prostate (The Korean Journal of Urology, vol. 483, 2007).

The principal symptoms caused by the obstructions include narrow stream, weak urine stream, hesitancy, intermittency, postvoid-dribbling or residual urine sense.

The treatment method is divided into prostatectomy and drug therapy. In the case that the symptoms are not enough serious for prostatectomy or patient favors non-operative treatment, or that the operative treatment is not proper for patient, the drug therapy is widely used.

Depending on the target site of drug, the drugs are divided into a drug to lower the mechanical obstruction by directly reducing the prostate size, and a drug to improve the dynamic obstruction by relaxing the smooth muscle in prostate and bladder neck.

The former group includes a drug of 5α-reductase inhibitors series and there are finasteride and dutasteride as typical drugs.

The latter group includes a drug of α-adrenergic receptor antagonist, and there are tamsulosin, alfuzosin, doxazosin, prazosin, indoramin, terazosin, silodosin and the like as typical drugs.

5α-reductase inhibitor takes three (3) months or longer to reduce the size of prostate by the drug work, and needs to be taken for about one year to show maximum effect. In addition, it can cause a sexual dysfunction such as erectile dysfunction.

α-adrenergic receptor antagonist improves rapidly the symptoms by acting on α-receptor and including relaxation of smooth muscle. However, it acts non-specifically other than the prostatic smooth muscle, thereby inducing the side effects such as swoon, a sense of lassitude, and sleepiness. Moreover, it has been reported that α-adrenergic receptor antagonist can cause a sexual dysfunction including retrograde ejaculation.

Incidence frequency of BPH, lower urinary tract symptoms, and erectile dysfunction is higher, as the age increases. Further, the more recent clinical studies report that the inhibitor of phosphodiesterase type 5 shows the treating efficacy of both BPH and lower urinary tract symptoms, it attracts attention as a medicine for the diseases (Urol Clin N Am 2005 32:511-525; BJU Int 2002 90:836-839; Expert Opin Drug Metab Toxicol 2006 2:609-617).

Particularly, in aspect of reduction of the side effect and increase of treatment effect of the existing drugs, the inhibitor of phosphodiesterase type 5 is noted as a medicine for BPH and lower urinary tract symptoms with increasing expectation.

On the one hand, the present inventors synthesized new pyrazolopyrimidinone compound represented by chemical formula (I), and disclosed a use of the compound as the inhibitor of phosphodiesterase type 5 (PDE 5 inhibitor) in WO 00/027848.

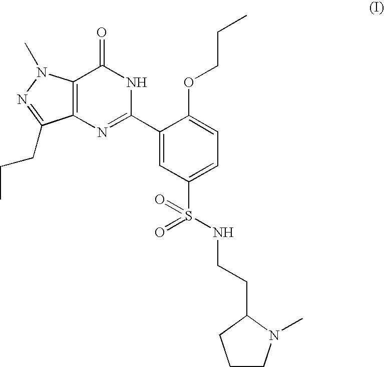

In addition, while the present inventors studied continuously the compound to be used as PDE 5 inhibitor, they found that the pyrazolopyrimidinone compound represented by chemical formula (I) showed excellent effect for treatment or prevention of BPH and lower urinary tract symptoms, and filed PCT application and Korean patent application (WO07/114534 and KR10-0792126B1).

Meanwhile, in combination of α-adrenergic receptor antagonist, some pyrazolopyrimidinone compounds used as PDE 5 inhibitors caused swoon by rapid drop in systemic blood pressure, and thus should be very careful when using with α-adrenergic receptor antagonist.

For example, when Vardenafil, one of existing PDE 5 inhibitors is used in combination of α-adrenergic receptor antagonist, some patients shows low blood pressure, and Vardenafil cannot be administered in 6 hours after administration of α-adrenergic receptor antagonist.

According to the result of recent clinical trial on drug interaction, when Vardenafil is administered after administering 10 mg of Terazocin and 0.4 mg of Tamsulosin, it shows average maximum reduction of a range between a maximum systolic pressure of 5-8 mmHg and a maximum diastolic pressure of of 4-6 mmHg. It is reported that when Terazocin or Tamsulosin is administered in combination of PDE 5 inhibitors, it shows a side effect of orthostatic hypotension.

### SUMMARY OF THE INVENTION

One embodiment of the present invention provides a combination therapy for BPH and LUTS.

Another embodiment of the present invention provides a composition and a method for treatment or prevention of BPH and LUTS, which has more excellent effect by relaxation of smooth muscle in prostate and bladder without showing side effect.

The present inventors found that the combinational administration of a specific pyrazolopyrimidinone compound as a PDE 5 inhibitor and α-adrenergic receptor antagonist shows better effect of treatment or prevention of BPH and LUTS, and gives higher safety in side effects such as drop of blood pressure and completed the present invention.

According to an embodiment of the present invention, provided is a composition for treatment or prevention of benign prostatic hyperplasia and lower urinary tract symptoms, comprising a pyrazolopyrimidinone compound represented by chemical formula (I) and α-Adrenergic receptor antagonist in an effective amount:

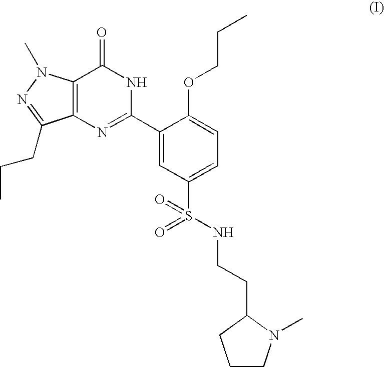

According to another embodiment, provided is a method of treating or preventing benign prostatic hyperplasia and lower urinary tract symptoms comprising administering to a patient in need thereof an effective amount of pyrazolopyrimidinone compound represented by chemical formula (I) and α-Adrenergic receptor antagonist.

### DETAILED DESCRIPTION

Before the present invention is disclosed and described, it should be understood that this invention is not limited to the particular configurations, process steps, and materials disclosed herein, and such configurations, process steps, and materials may be varied. It should be also understood that the terminology employed herein is used for the purpose of describing particular embodiments only and is not intended to limit the scope of the present invention which will be limited only by the appended claims and equivalents thereof.

Unless explicitly described to the contrary, the term “comprise” and variations such as “comprises” or “comprising”, “include” and “including' will be understood to include any constituent element (or constituent component) without limitation but not to exclude other constituent element (or constituent component) not explicitly set forth.

As used herein, the term “combinational administration” and variations such as “combination therapy” and “administered in combination with” means, for example, administering simultaneously a pyrazolopyrimidinone compound and α-Adrenergic receptor antagonist in same composition including a pyrazolopyrimidinone compound represented by chemical formula (I) and α-Adrenergic receptor antagonist or in separate compositions including a pyrazolopyrimidinone compound and α-Adrenergic receptor antagonist respectively, and means, for example, administering sequentially a pyrazolopyrimidinone compound and α-Adrenergic receptor antagonist at an interval that two active agents stay together in the body

According to the embodiments of the present invention, provided are a composition and a method for treatment or prevention of benign prostatic hyperplasia and lower urinary tract symptoms,

The composition comprises (5-[2-propyloxy-5-(1-methyl-2-pyrolidinylethylamidosulphonyl)phenyl]-1-methyl-propyl-1,6-dihydro-7H-pyrazolo (4,3-d)pyrimidin-7-one) represented by chemical formula (I) and α-Adrenergic receptor antagonist in an effective amount.

The method of treatment or prevention is a combinational administration of a pyrazolopyrimidinone compound represented by chemical formula (I) and α-Adrenergic receptor antagonist in an effective amount:

As fully supported by following exemplary embodiments, the present inventors showed that the combinational administration of the pyrazolopyrimidinone compound of chemical formula (I) as a PDE 5 inhibitor and α-Adrenergic receptor antagonist represented better effect of treatment or prevention of BPH and LUTS by synergistic effect, and gives higher safety in side effects such as drop of blood pressure.

The drugs, for example, reduce synergistically urethral pressure by relaxation of smooth muscle in prosthetic and urinary smooth muscle, and thus show excellent effect of treatment or prevention for BPH and LUTS.

Accordingly, the composition and the method can be properly used for treatment or prevention of BPH and LUTS.

The pyrazolopyrimidinone compound represented by chemical formula (I) inhibits PDE 5 strongly with high selectivity as one of PDE 5 inhibitors, and shows improved solubility, rapid absorption, high bioavailability and high distribution volume in the body.

Particularly, the pyrazolopyrimidinone compound is characterized in three times longer half life in the body, in comparison of 1-{[3- (6,7-dihydro-1-methyl-1-7-oxo-3-propyl-1H-pyrazolo-[4,3-d]pyrimidin-5-ily)-4-ethoxyphenyl]sulfonyl} citrate or 1-{[3-(1,4-dihydro-5-methyl-4-oxo-7-propylimidazo[5,1-f][1,2,4] triazin-2-ily)-4-ethoxyphenyl]sulfonyl-4-piperazine mono hydrochloride with the same drug mechanism.

The pyrazolopyrimidinone compound has 158-161° C. of melting point, and about 6.5 of pKa₁ and about 12.5 of pKa₂, low solubility to water but high solubility to acetic acid, methanol or chloroform, etc. and is in a form of white or yellowish-white powder but not hydrate or solvate.

The pyrazolopyrimidinone compound represented by chemical formula (I) can be prepared by three synthesizing steps, as disclosed in WO 00/027848 and Korean Patent No. 0353014.

An exemplary preparation method of the pyrazolopyrimidinone compound is described in detail.

Firstly, 442-propyloxy-5-(chlorosulfonyl)benzamido]-1-methyl-3-propyl-5-carbamoyl pyrazole is prepared, for example, by adding an amount of 4-[2-propyloxylbenzamido]-1-methyl-3-propyl-5-carbamoyl pyrazole to an amount of chlorosulfonic acid cooled to 0° C., agitating the reaction mixture, filtering, washing and drying.

Then, 4-[2-propyloxy-5-(1-methyl-2-pyrolidinylethylamidosulfonyl) benzamido]-1-methyl-3-propyl-5-carbamoyl pyrazole is synthesized from the reaction product. For example, at 0° C., an amount of 442-propyloxy-5-(chlorosulfonyl)benzamido]-1-methyl-3-propyl-5-carbamoyl pyrazole dissolved in dichloromethane is agitated with addition of 2-(2-aminoethyl)-1-methyl pyrolidine. After the reaction is terminated, the resultant solution is diluted with dichloromethane, and the organic solvent layer is taken, washed, dried, concentrated and filtered to obtain 4-[2-propyoxy-5-(1-methyl -2-pyrolidinylethyl amindosulfonyl)benzamindo]-1-methyl-3-propyl-5-carbamoyl pyrazole.

The pyrazolopyrimidinone compound of chemical formula (I) is prepared by dissolving 4-[2-propyoxy-5-(1-methyl-2-pyrolidinylethyl amindosulfonyl) benzamindo]-1-methyl-3-propyl-5-carbamoyl pyrazole in t-butanol, and agitating with addition of potassium t-butoxide. After the reaction is terminated, the resultant solution is performed by cooling, diluting, washing, drying, distilling under vacuum, removing the solvent, and silica gel column chromatography to obtain the compound of chemical formula (I).

In the composition and the method according to the embodiments of the present invention, for example, the compound of chemical formula (I) is included in a composition together with α-Adrenergic receptor antagonist, or is administered in combination with α-Adrenergic receptor antagonist for treatment or prevention of BPH and LUTS, as described in detail.

In case that the pyrazolopyrimidinone compound of chemical formula (I) is included in a composition together with α-Adrenergic receptor antagonist, or is administered in combination with α-Adrenergic receptor antagonist, it reduces largely side effect (for example, drop in blood pressure or sexual dysfunction including retrograde ejaculation, etc.) caused by drug interaction, and dramatically improves relaxation of smooth muscle in prostate and bladder by NO cGMP pathway and α-Adrenergic receptor antagonist, compared to administration of each drug alone. Accordingly, the combinational administration of the pyrazolopyrimidinone compound of chemical formula (I) and α-Adrenergic receptor antagonist shows excellent effect of treatment or prevention for BPH and LUTS

In comparison of conventional pharmaceutical composition or method of treatment or prevention, the composition and the method of the present invention are effectively applied for treatment or prevention of BPH and LUTS.

The pyrazolopyrimidinone compound of chemical formula (I) can be included in a composition together with α-Adrenergic receptor antagonist, or is administered in combination with α-Adrenergic receptor antagonist, where the α-Adrenergic receptor antagonist is any one well known to a ordinary person skilled in the art.

The examples of α-Adrenergic receptor antagonist can be any one used for treating or preventing prostatic hypertrophy, such as (R)-5-(2-(2-(2-ethoxyphenoxy)ethylamino)propyl)-2-methoxy benzensulfonamide (tamsulosin) or 1-(3-hydroxypropyl)-5-{2R-2-[2-(2,2,2-trifluoroethoxy)phenoxy]ethylamino]propyl}-7-indolincarboxamide (silodosin) and a mixture thereof, but not limited thereto.

The composition is formed in various formulations to be administered orally, enterally, parenterally, and etc. and the formulations are obtained according to well-known method and constituents which are known to a person skilled in the art. Preferably, the composition is formed in an oral formulation such as a capsule or table.

In addition, the composition further includes diluents or excipients such as widely-used filler, extender, binder, wetting agent, disintegrant, and surfactant depending on the formulations.

The amount of active agent and administration and dosage applied for the composition and the method of the embodiments can be selected properly by a person skilled in the art depending on body weight, age, sex, health condition, diet, administration period, administration method, excretory rate, and seriousness of disease.

For example, the composition may comprises 25 to 200 mg, preferably 25 to 100 mg, more preferably 50 to 100 mg of the pyrazolopyrimidinone compound of chemical formula (I) and 0.1 to 50 mg, preferably 0.1 to 25 mg, more preferably 0.1 to 10 mg of the α-Adrenergic receptor antagonist.

Also, according to the method of the embodiment, in reference to an adult with 70 kg body weight, the dose administered per day may be 25 to 200 mg/day, preferably 25 to 100 mg/day, more preferably 50 to 100 mg/day of the pyrazolopyrimidinone compound and 0.1 to 50 mg/day, preferably 0.1 to 25 mg/day, more preferably 0.1 to 10 mg/day of the α-Adrenergic receptor antagonist in combination. The dose may be administered once or several times a day.

Further, the unit dosage form of the composition can include 25 to 200 mg/day, preferably 25 to 100 mg/day, more preferably 50 to 100 mg/day of the pyrazolopyrimidinone compound and 0.1 to 50 mg/day, preferably 0.1 to 25 mg/day, more preferably 0.1 to 10 mg/day of the α-Adrenergic receptor antagonist. In addition, it is well-known to a person skilled in the art from the above description that the compositions including the active agents at various amounts and unit dosage form thereof can be formed according to an administered amount per unit time interval applied by the composition (for example, 6 hours, 12 hours, 1 day, or 2 days).

As aforementioned, when the composition and the method of the embodiments comprise a proper amount or dosage of the active agents in combination, they can show better effect of treatment or prevention for BPH and LUTS with a less amount or dosage of the active agents and give higher safety in side effects such as drop of blood pressure or sexual dysfunction including retrograde ejaculation.

The present invention is further explained in more detail with reference to the following examples. These examples, however, should not be interpreted as limiting the scope of the present invention in any manner.

### Example 1

Evaluation for Effect of Pyrazolopyrimidinone Compound Represented by Chemical Formula (I) and α-Adrenergic Receptor Antagonist on the Relaxation of Smooth Muscle of Rat Prostate

When pyrazolopyrimidinone compound represented by chemical formula (I) and (R)-5-(2-(2-(2-ethoxyphenoxy)ethylamino)propyl)-2-methoxy benzensulfonamide (Tamsulosin) as α-Adrenergic receptor antagonist were used alone or in combination, their respective effect on the relaxation of smooth muscle of rat prostate were evaluated and compared according to the following experiments.

Male Sprague-Dawley rat with 300-400 g of body weight was sacrificed and the prostate was extracted and transferred to Krebs-Henseleit buffer kept cold. The dorsolateral lobes of prostatic tissue were transected to separate prostatic tissue gland with size of 2×10 mm (4 tissues per a rat).

The separated tissues were transferred to organ bath where 95% O₂, and 5% CO₂ were emitted and Krebs-Henseleit buffer (37° C.) was contained, both ends of the tissue gland were fixed to the bath floor and force transducer connected to polygraph(Grass Instruments) respectively. Each tissue was stabilized under 0.2 standard tension for 1 hour, and then treated by 1 μM of phenylephrine to induce the contraction of prostatic tissue. After the contraction was stable, the pyrazolopyrimidinone of formula (I) were treated alone or in combination with Tamsulosin, and then carried out by measurement of the contraction degree (Table 1) and evaluation of the relaxation degree (inverse proportion to the contraction degree).

TABLE 1
Evaluation of effect on the relaxation of rat prostatic tissue

| | Compound of | | | | | | |
|---|---|---|---|---|---|---|---|
| | formula (I) | | | Tamsulosin | | | |
| | | | treat- | | | treat- | Contraction |
| | concentration | | ment | concentration | | ment | degree (%) |
| test 1 | 0 | μM | no | 0 | nM | no | 100 |
| test 2 | 3 | μM | Yes | 0 | nM | no | 92.6 ± 2.2 |
| test 3 | 10 | μM | Yes | 0 | nM | no | 77.1 ± 2.5 |
| test 4 | 30 | μM | Yes | 0 | nM | no | 62.2 ± 3.1 |
| test 5 | 0 | μM | no | 0.25 | nM | yes | 90.8 ± 1.4 |
| test 6 | 3 | μM | Yes | 0.25 | nM | yes | 71.5 ± 9.4 |
| test 7 | 10 | μM | Yes | 0.25 | nM | yes | 47.7 ± 14.7 |
| test 8 | 30 | μM | Yes | 0.25 | nM | yes | 39.6 ± 9.4 |
| test 9 | 0 | μM | no | 1 | nM | yes | 78.8 ± 2.0 |
| test 10 | 3 | μM | Yes | 1 | nM | yes | 41.8 ± 5.7 |
| test 11 | 10 | μM | Yes | 1 | nM | yes | 32.0 ± 14.9 |
| test 12 | 30 | μM | Yes | 1 | nM | yes | 28.1 ± 4.8 |
| test 13 | 0 | μM | no | 4 | nM | yes | 43.6 ± 4.2 |
| test 14 | 3 | μM | Yes | 4 | nM | yes | 23.4 ± 4.4 |
| test 15 | 10 | μM | Yes | 4 | nM | yes | 12.3 ± 17.4 |
| test 16 | 30 | μM | Yes | 4 | nM | yes | 6.1 ± 4.0 |

In Table 1, data were obtained by using four (4) tissue glands were used for each concentration of the drug, and the numerical value is mean ±standard error of the percentage of contracting degree (inverse proportion to percentage of relaxation degree).

As shown in Table 1, in comparison with administration of either the pyrazolopyrimidinone compound of formula (I) or Tamsulosin alone, the combinational administration thereof represented synergistic effect on the relaxation of smooth muscle in prostate.

Accordingly, when the combination including the pyrazolopyrimidinone compound of formula (I) and Tamsulosin, and the method of combinational administration were applied, the synergistic effect on relaxation of smooth muscle of prostate. The result confirmed that the combinational administration gave excellent effect of treatment or prevention for BPH and LUTS.

### Example 2

Evaluation for Effect of Pyrazolopyrimidinone Compound Represented by Chemical Formula (I) and α-Adrenergic Receptor Antagonist on the Relaxation of Smooth Muscle of Rat Bladder

In case of the administration of pyrazolopyrimidinone of formula (I) alone, pre-treatment of N-nitro-L-arginine methyl ester (L-NAME) as nitric oxide inhibitor, and the combinational administration of pyrazolopyrimidinone of formula (I) and Tamsulosin as α-Adrenergic receptor antagonist, the effect of the treatments on the relaxation of smooth muscle of rat bladder were evaluated and compared according to the following experiments.

Male Sprague-Dawley rat with 200-250 g of body weight was sacrificed and the prostate was extracted and transferred to Krebs-Henseleit buffer kept cold. The bladder was cut vertically with size of 2 mm and transferred to organ bath where 95% O₂, and 5% CO₂ were emitted and Krebs-Henseleit buffer (37° C.) was contained, both ends of the tissue gland were fixed to the bath floor and force transducer connected to polygraph(Grass Instruments) respectively. Each tissue was stabilized for 1 hour, and then treated by 10⁻⁵M carbachol to induce the contraction of prostatic tissue. After the contraction was stable, the tissue were carried out by the administration of pyrazolopyrimidinone (10⁻⁵M to 10⁻⁴M) of formula (I) alone, pre-treatment of L-NAME (10⁻⁴M) as nitric oxide inhibitor, and the combinational administration of pyrazolopyrimidinone of formula (I) and Tamsulosin (10⁻⁴M) as α-Adrenergic receptor antagonist, and then performed by measurement and evaluation of the relaxation degree (Table 2).

TABLE 2
Evaluation of effect on the relaxation of rat bladder tissue

| | Concentration of Compound of formula (I) | | | | |
|---|---|---|---|---|---|
| Treatment | 10⁻⁸M | 10⁻⁷M | 10⁻⁶M | 10⁻⁵M | 10⁻⁴M |
| Compound of formula (I) | 0.0 ± 0.0 | 0.0 ± 0.0 | 2.0 ± 1.1 | 7.2 ± 1.6 | 17.4 ± 3.7 |
| L-NAME (10⁻⁴M) and | 0.0 ± 0.0 | 0.0 ± 0.0 | 0.0 ± 0.0 | 0.0 ± 0.0 | 0.0 ± 0.0 |
| Compound of formula (I) | | | | | |
| Tamsulosin (10⁻⁴M) and | 13.0 ± 1.1 | 13.0 ± 0.9 | 15.0 ± 1.3 | 20.1 ± 3.6 | 32.3 ± 3.2 |
| Compound of formula (I) | | | | | |

In Table 2, data were obtained by using four (4) tissue glands were used for each concentration of the drug, and the numerical value is mean ± standard error of the percentage of relaxation degree.

As shown in Table 2, when the pyrazolopyrimidinone of formula (I) was administered alone, the relaxation effect began to be represented at a concentration of 10⁻⁶M, and was about 17.4% relaxation degree at a concentration of 10⁻⁴M.

On the other hand, when the pyrazolopyrimidinone of formula (I) was treated after treatment of Tamsulosin as a representative α-adrenergic receptor antagonist, the relaxation of smooth muscle of rat bladder was increased largely at each concentration of the pyrazolopyrimidinone. In addition, the pre-treatment of L-NAME as nitric oxide inhibitor removed the relaxation effect of the pyrazolopyrimidinone on the bladder smooth muscle.

Accordingly, when the combination including the pyrazolopyrimidinone of chemical formula (I) and Tamsulosin, and the method of combinational administration were applied, the synergistic effect on relaxation of smooth muscle of bladder. The result confirmed that the combinational administration induced an excellent effect of treatment or prevention for BPH and LUTS.

### Example 3

Evaluation for Effect of Pyrazolopyrimidinone Compound Represented by Chemical Formula (I) and α-Adrenergic Receptor Antagonist on the Relaxation of Smooth Muscle of Rat Prostate

When pyrazolopyrimidinone compound represented by chemical formula (I) and 1-(3-hydroxypropyl)-5-{2R-2-[2-(2,2,2-trifuloroethoxy)phenoxy]ethylamino]propyl}-7-indolincarboxamide (Silodosin) as α-Adrenergic receptor antagonist were used alone or in combination, their respective effect on the relaxation of smooth muscle of rat prostate were evaluated and compared according to the following experiments.

Male Sprague-Dawley rat with 300-400 g of body weight was sacrificed and the prostate was extracted and transferred to Krebs-Henseleit buffer kept cold. The dorsolateral lobes of prostatic tissue were transected to separate prostatic tissue gland with size of 2×10 mm (4 tissues per a rat). The separated tissues were transferred to organ bath where 95% O₂, and 5% CO₂ were emitted and Krebs-Henseleit buffer (37° C.) was contained, both ends of the tissue gland were fixed to the bath floor and force transducer connected to polygraph(Grass Instruments) respectively.

Each tissue was stabilized for 1 hour, and then treated by 10⁻⁵M carbachol to induce the contraction of prostatic tissue. After the contraction was stable, the pyrazolopyrimidinone of chemical formula (I) were treated alone at a concentration of 3×10⁻⁶M, 10⁻⁵M, and 3×10⁻⁵M, or in combination with Silodosin (0.1 nM, 1 nM, and 10 nM) as α-Adrenergic receptor antagonist, and then performed by measurement of the contraction degree (Table 3) and evaluation of the relaxation degree (inverse proportion to the contraction degree).

As shown in Table 3, when the pyrazolopyrimidinone of chemical formula (I) was administered in combination with Silodosin as a representative α-Adrenergic receptor antagonist at various concentrations, the synergistic effect on relaxation of smooth muscle of prostate were confirmed.

TABLE 3
Evaluation of effect on the relaxation of rat prostatic tissue

| | Compound of | | | | | | |
|---|---|---|---|---|---|---|---|
| | formula (I) | | | Silodosin | | | |
| | | | treat- | | | treat- | Contraction |
| | concentration | | ment | concentration | | ment | degree (%) |
| test 1 | 0 | μM | No | 0 | nM | no | 100 |
| test 2 | 3 | μM | Yes | 0 | nM | no | 92.6 ± 2.2 |
| test 3 | 10 | μM | Yes | 0 | nM | no | 77.1 ± 2.5 |
| test 4 | 30 | μM | Yes | 0 | nM | no | 62.2 ± 3.1 |
| test 5 | 0 | μM | No | 0.1 | nM | yes | 90.8 ± 1.4 |
| test 6 | 3 | μM | Yes | 0.1 | nM | yes | 71.5 ± 9.4 |
| test 7 | 10 | μM | Yes | 0.1 | nM | yes | 47.7 ± 14.7 |
| test 8 | 30 | μM | Yes | 0.1 | nM | yes | 39.6 ± 9.4 |
| test 9 | 0 | μM | No | 1 | nM | yes | 82.0 ± 1.7 |
| test 10 | 3 | μM | Yes | 1 | nM | yes | 56.6 ± 4.2 |
| test 11 | 10 | μM | Yes | 1 | nM | yes | 44.7 ± 4.1 |
| test 12 | 30 | μM | Yes | 1 | nM | yes | 38.8 ± 4.7 |
| test 13 | 0 | μM | No | 10 | nM | yes | 46.9 ± 1.6 |
| test 14 | 3 | μM | Yes | 10 | nM | yes | 19.8 ± 6.6 |
| test 15 | 10 | μM | Yes | 10 | nM | yes | 13.2 ± 4.1 |
| test 16 | 30 | μM | Yes | 10 | nM | yes | 3.2 ± 1.4 |

In Table 3, data were obtained by using four (4) tissue glands were used for each concentration of the drug, and the numerical value is mean ± standard error of the percentage of contracting degree (inverse proportion to relaxation degree).

### Example 4

### Safety Test 1

28 health men aged 19 to 50 years were divided into four groups according to randomization list, and administered by a) placebo and placebo (Group A), b) placebo and the pyrazolopyrimidinone of chemical formula (I) (Group B), c) Tamsulosin and placebo (Group C) and d) Tamsulosin and the pyrazolopyrimidinone of chemical formula (I) (Group D) to evaluate the effect of the combinational administration of the pyrazolopyrimidinone of chemical formula (I) and Tamsulosin on the safety.

The safety evaluation was performed by observing vital signs (diastolic blood pressure, pulse rate, body temperature), clinical laboratory testing, electrocardiogram, adverse reactions such as subjective symptom and objective symptom. In addition, the effect of drug interaction on pharmacokinetics of the pyrazolopyrimidinone was observed. The results were shown in Tables 4 and 5.

TABLE 4
Test result of the vital signs in supine position

| | | Group A | Group B | Group C | Group D |
|---|---|---|---|---|---|
| Diastolic blood | mean | 116.0 | 112.4 | 112.3 | 112.1 |
| pressure | CV(%) | 9.6 | 8.9 | 8.6 | 8.2 |
| Systolic blood | mean | 64.5 | 61.6 | 63.0 | 61.4 |
| pressure | CV(%) | 11.1 | 10.9 | 10.7 | 9.8 |
| Pulse rate | mean | 58.9 | 60.2 | 57.7 | 64.9 |
| | CV(%) | 12.9 | 13.4 | 12.7 | 14.1 |

Group A: placebo + placebo
Group B: placebo + the pyrazolopyrimidinone of formula (I)
Group C: placebo + Tamsulosin
Group D: Tamsulosin + the pyrazolopyrimidinone of formula (I)
CV: coefficient of variation

The clinical test result of the vital signs in supine position confirmed that the combinational administration of the pyrazolopyrimidinone of chemical formula (I) and Tamsulosin, the reduction of average systolic and diastolic blood pressures were not observed in clinical significance, compared to that of the administration of Tamsulosin alone.

As a result of comparison with Cₘₐₓ, AUC_{inf}, Tₘₐₓ and T_{1/2} of Group C and Group D, two groups did not show the statistically-significant difference.

TABLE 5
Pharmacokinetic parameters

| | Tₘₐₓ (h) | Cₘₐₓ | AUC_{inf} | T_{1/2} (h) |
|---|---|---|---|---|
| Group B | 1.5 | 617.0 ± 169.1 | 4726.9 ± 1263.1 | 9.7 ± 1.7 |
| Group D | 1.1 | 623.7 ± 199.6 | 4911.3 ± 1260.3 | 9.9 ± 2.3 |

Tₘₐₓ: Time to reach a maximum plasma concentration after the administration
Cₘₐₓ: Maximum plasma concentration after the administration
AUC_{inf}: Area under the plasma concentration versus time curve which is calculated by extrapolation of unlimited time as follow:
AUC_{inf} = AUCₗₐₛₜ + Cₗₐₛₜ/λ_{z}
AUCₗₐₛₜ: Area under the plasma concentration-time curve which is calculated at measurable final point of time according to trapezoidal method. Specifically, the area is calculated according to the linear trapezoidal method at region of increasing plasma concentration, and according to log-linear trapezoidal sum method at region of decreasing plasma concentration, with excluding the concentration value of less than LLOQ.
Cₗₐₛₜ: plasma concentration at measurable final point of time.
λ_{z}, t_{1/2}: elimination rate constant (λ_{z}) and half life(t_{1/2}). Half life is calculated from ln(2)/λ_{z} and elimination rate constant is obtained from linear regression analysis of the terminal log-linear region of the plasma concentration-time curve.

The safety and pharmacokinetics results confirmed that compared to the administration of the pyrazolopyrimidinone alone (Group B), the combinational administration of Tamsulosin and the pyrazolopyrimidinone (Group D) did not cause the significant difference in safety change and pharmacokinetics result.

### Example 5

### Safety Test 2

9 health men aged 19 to 50 years were divided into three groups according to randomization list, and administered by a) placebo and the pyrazolopyrimidinone of chemical formula (I) (Group A), b) placebo and Silodosin (Group B), and c) Silodosin and the pyrazolopyrimidinone of formula (I) (Group C) to evaluate the effect of the combinational administration of the pyrazolopyrimidinone of formula (I) and Silodosin on the safety.

The safety evaluation was performed by observing vital signs (diastolic blood pressure, pulse rate, body temperature), clinical laboratory testing, electrocardiogram, adverse reactions such as subjective symptom and objective symptom. In addition, the effect of drug-drug interaction on pharmacokinetics of the pyrazolopyrimidinone was observed. The results were shown in Tables 6 and 7.

TABLE 6
Test result of the vital signs in supine position

| | | Group A | Group B | Group C |
|---|---|---|---|---|
| Diastolic blood | mean | 112.8 | 112.4 | 112.2 |
| pressure e | CV (%) | 8.5 | 8.8 | 8.4 |
| Systolic blood | mean | 62.2 | 62.3 | 61.7 |
| pressur | CV (%) | 9.5 | 10.5 | 10.1 |
| Pulse rate | mean | 61.4 | 64.2 | 61.5 |
| | CV (%) | 12.4 | 12.7 | 13.1 |

Group A: placebo + the pyrazolopyrimidinone of formula (I)
Group B: placebo + Silodosin
Group C: Silodosin + the pyrazolopyrimidinone of formula (I)
CV (coefficient of variation)

The clinical test result of the vital signs in supine position confirmed that the combinational administration of the pyrazolopyrimidinone and Silodosin, the reduction of average systolic and diastolic blood pressures were not observed in clinical significance, compared to that of the administration of Silodosin alone.

TABLE 7
Pharmacokinetic parameters

| | Tₘₐₓ (h) | | Cₘₐₓ | | AUC_{inf} | |
|---|---|---|---|---|---|---|
| | Ud | Silodosin | Ud | Silodosin | Ud | Silodosin |
| Group A | 1.5 | — | 627.2 ± 158.5 | — | 4822.9 ± 1323.2 | — |
| Group B | — | 1.4 | — | 31.1 ± 7.2 | — | 110.2 ± 17.6 |
| Group C | 1.3 | 1.7 | 659.5 ± 195.7 | 35.2 ± 7.8 | 4942.2 ± 1258.5 | 132.5 ± 25.2 |

* Ud: the pyrazolopyrimidinone compound of chemical formula (I)
Tₘₐₓ: Time to reach a maximum plasma concentration after the administration
Cₘₐₓ: Maximum plasma concentration after the administration
AUC_{inf}: Area under the plasma concentration versus time curve which is calculated by extrapolation of unlimited time as follow:
AUC_{inf} = AUCₗₐₛₜ + Cₗₐₛₜ/λ_{z}
AUCₗₐₛₜ: Area under the plasma concentration-time curve which is calculated at measurable final point of time according to trapezoidal method. Specifically, the area is calculated according to the linear trapezoidal method at region of increasing plasma concentration, and according to log-linear trapezoidal sum method at region of decreasing plasma concentration, with excluding the concentration value of less than LLOQ.
Cₗₐₛₜ: plasma concentration at measurable final point of time.

As a result of pharmacokinetics results, Cₘₐₓ, AUC_{inf}, Tₘₐₓ and T_{1/2}, did not show the statistically-significant difference.

The safety and pharmacokinetics results confirmed that compared to the administration of the Silodosin or pyrazolopyrimidinone alone, the combinational administration of Silodosin and the pyrazolopyrimidinone did not induce the significant difference in safety change and pharmacokinetics result.

What is claimed is:

1. A composition for treatment or prevention of benign prostatic hyperplasia and lower urinary tract symptoms, comprising a pyrazolopyrimidinone compound represented by chemical formula (I) and α-Adrenergic receptor antagonist in an effective amount:

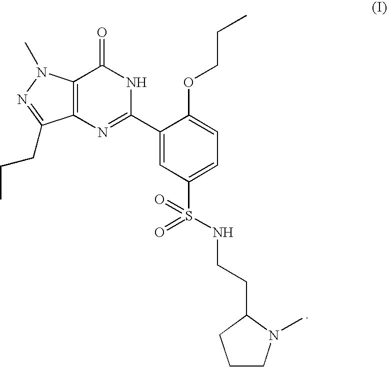

2. The composition of claim 1, wherein the α-Adrenergic receptor antagonist comprises (R)-5-(2-(2-(2-ethoxylphenoxy)ethylamino)propyl)-2-methoxy benzensulfonamide.

3. The composition of claim 1, wherein the α-Adrenergic receptor antagonist comprises 1-(3-hydroxypropyl)-5-{2R-2-[2-(2,2,2-trifluoroethoxy)phenoxy]ethylamino]propyl}-7-indolincarboxamide.

4. The composition of claim 1, wherein the composition lowers urethral pressure by relaxation of smooth muscle in prostate and bladder.

5. The composition of claim 1, wherein the composition comprises 25 to 200 mg of the pyrazolopyrimidinone compound and 0.1 to 50 mg of the α-Adrenergic receptor antagonist.

6. A method of treating or preventing benign prostatic hyperplasia and lower urinary tract symptoms comprising administering to a patient in need thereof an effective amount of pyrazolopyrimidinone compound represented by chemical formula (I) and α-Adrenergic receptor antagonist:

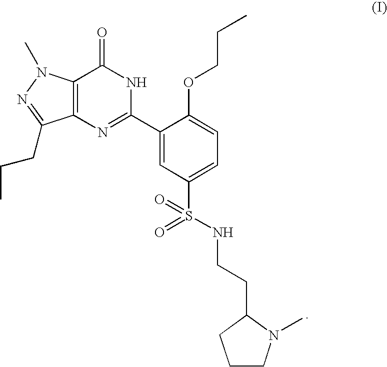

7. The method of claim 6, wherein the pyrazolopyrimidinone compound and α-Adrenergic receptor antagonist are administered sequentially or simultaneously.

8. The method of claim 6, wherein the α-Adrenergic receptor antagonist comprises (R)-5-(2-(2-(2-ethoxylphenoxy)ethylamino)propyl)-2-methoxy benzensulfonamide.

9. The method of claim 6, wherein the α-Adrenergic receptor antagonist comprises 1-(3-hydroxypropyl)-5-{2R-2-[2-(2,2,2-trifluoroethoxy)phenoxy]ethylamino]propyl}-7-indolincarboxamide.

10. The method of claim 6, wherein after administering the pyrazolopyrimidinone compound and the α-Adrenergic receptor antagonist, urethral pressure is lowered by relaxation of smooth muscle in prostate and bladder.

11. The method of claim 6, wherein the pyrazolopyrimidinone compound is administered in a dose of 25 to 200 mg/day and the α-Adrenergic receptor antagonist is administered in a dose of 0.1 to 50 mg/day.
